(19) United States
(12) Patent Application Publication
(10) Pub. No.: US 2010/0016784 A1
(43) Pub. Date: Jan. 21, 2010

(54) POSITIONABLE MEDICAL SYSTEM FOR POSITIONING MEDICAL COMPONENTS ON OR WITHIN A BODY

(75) Inventors: Dinesh I. MODY, Pleasanton, CA (US); Dany Berube, Fremont, CA (US); Ketan SHROFF, Pleasanton, CA (US)

Correspondence Address: LEVINE BAGADE HAN LLP, 2400 GENG ROAD, SUITE 120, PALO ALTO, CA, 94303, US

(73) Assignee: MicroCube LLC, Fremont, CA (US)

(21) Appl. No.: 12/175,313

(22) Filed: Jul. 17, 2008

(51) Int. Cl. A61B 18/18; A61H 1/00; A61B 18/20; A61B 18/02

(52) U.S. Cl. 604/22; 606/41; 601/2; 606/13; 606/21

(57) ABSTRACT

Devices, systems and methods relating to delivery systems suitable for positioning various medical components within a patient's body and, more specifically, to delivery systems designed to cooperate with certain therapeutic and diagnostic devices.

### CROSS-REFERENCE TO RELATED APPLICATIONS

n/a

### FIELD OF THE INVENTION

This invention relates to delivery systems suitable for positioning various medical components within a patient's body and, more specifically, to delivery systems designed to cooperate with certain therapeutic and diagnostic devices.

### BACKGROUND OF THE INVENTION

Medical diagnostic and surgical procedures generally require that physicians perform many increasingly complex functions within the body. These physicians must, properly access tissue locations and orient a medical component to perform a diagnostic or surgical procedure. In an ever increasing number of modern surgical procedures, physicians deliberately keep openings, which provide access into the body, small to minimize the trauma and the healing burden on the patient. For example, in many cardiac procedures, a surgeon will either access internal cardiac tissue by navigating a device through the vasculature until the device can engage the cardiac tissue (e.g., navigating a device through vasculature until the device is located within a chamber of the heart). In another example, a physician may create an opening (or rely upon a natural body opening) to access organs and tissues requiring a medical procedure.

Traditionally, the desire to minimize trauma to the patient often comes at the expense of the maneuverability of the medical component when compared to a similar open surgical procedure. For instance, a physician working with his hands and having full access to a target site has the ability to perform many functions with predictability and stability. Yet, such maneuverability is lost during minimally invasive procedures or even those open surgical procedures where the physician's access to the target site is limited due to obstructing tissue or organs (such as accessing the interior of an organ through a wall of the organ).

Many conventional approaches attempt to overcome these limitations on maneuverability by providing a device with a steerable distal end. However, these devices are limited since the optimal amount of articulation and/or manipulation needed within the body is difficult, or impossible using conventional devices, especially when there is indirect visual access. Moreover, conventional steerable devices are simply no substitute for control and precision afforded by the use of the physician's hands.

To illustrate this point, consider the situation in an open surgical procedure, where a physician is able to use both arms to directly manipulate a treatment device onto or along a body structure. While the numerous degrees of freedom offered by a single arm (including the hand and fingers) enables satisfactory placement of the treatment device, the synergistic effect of using both arms makes placement of the treatment device even more precise and efficient. When the physician attempts the same procedure through a limited access (either a smaller incision, use of access ports, intravascularly or even through dissection of other tissues/organs), conventional steerable devices do not provide the synergistic effects of using both arms. Conventional steerable devices do not offer true synergistic effects when coupled with a second steerable device as such devices are designed to work in a stand alone mode.

In view of the above, there remains a need for a device or system that offers improved positioning in various medical procedures. There also remains a need for a device or system that allows a physician the capability to position an appropriate medical component readily and predictably at all desired locations (where the locations may be 2 or 3 dimensional structures) for performing the required procedure.

### SUMMARY OF THE INVENTION

The systems and methods described herein are useful in a variety of medical procedures including various diagnostic and surgical treatments. In addition, the invention is also useful in open surgical procedures, minimally invasive procedures, as well as procedures performed through natural body openings.

The systems and methods can be used in such medical procedures ranging from endovascular cardiac, thoracic cardiac, bronchial, lung, gynecological, gastro-intestinal, spinal, urinary, ENT, laparoscopic, intracranial, intra-peritoneal, thoracoscopic, bronchoscopic, cystoscopy colposeopie, hysteroscopic, arthroscopic, etc.

In a first variation, the invention includes a medical system for performing a medical procedure on or in a patient, the system comprising at least a first and second arm, each arm having a maneuverable distal portion and a proximal portion, where manipulation of the respective arm's proximal portion permits articulation of the arm's maneuverable distal portion independently of the other respective arm, and a first medical component coupled to the first arm and extendable from the first arm's maneuverable distal portion, the first medical component being engageable with the second arm's maneuverable distal portion, such that when coupled to both arms, movement of either maneuverable distal portion alters a profile or position of the first medical component allowing for positioning of the first medical component to perform the medical procedure.

Although many variations of the device are discussed and/or illustrated with two arms, variations of the device include any number of arms. In some variations, the medical components extend through the arm in a concentric or coaxial manner. However, the invention also includes components that extend parallel to the arm, or that extend along the arm or a portion thereof in a non-concentric manner. The arms can be removably coupleable to the medical components) via a grasping structure. An example of such grasping structure includes a releasable hook, ring, or jaws (where such structures are well known by those skilled in the art. The arms can fully or partially hollow (so long as they allowing for a coupling portion to couple the medical component). One or more arms may be concentric with another. They may be introduced via one or more access ports and pathways.

For purposes of this specification, the term medical component is intended to include a medical device or portion thereof that is adapted to provide a visual, diagnostic, or treatment procedure. For example, and as described below, in one variation, the system can include a first component comprising an energy delivery device configured to deliver energy (e.g., radiofrequency, DC, microwave, ultrasound, laser, cryogenic energy) to or from tissue and a second component (such as a guide wire, rail, tether, flexible member, etc.) that is used to direct the energy delivery device towards a target site. The first and second medical component can be parts of the same device or can be discrete medical devices.

Examples of medical components, include, but are not limited to, therapeutic devices such as ablation devices for imparting a treatment to a target tissue, diagnostic devices such as mapping catheters for providing physiological information regarding a target tissue; positioning devices which include elements for providing additional positioning of additional functional devices (e.g., guidewires, rails, tethers, introducer catheters, sheaths, etc.), imaging devices, or non-imaging feedback devices (such as a Doppler catheter). The component need not have a specific physical structure, for example the arms of the inventive system can be adapted to deploy a simple tube that administers a chemical ablating agent at a desired location or deploy an additional fluid used during, and in support of, the medical procedure, for example deployment of contrast agent to provide a clearer view of the anatomy in support, of a procedure performed within a patient's heart. In yet additional variations, the medical components can include separate components used to provide a single diagnostic procedure or different steps of the same medical procedure. For instance, when using a radiofrequency energy modality, the first medical component could include a first electrode while the second component can include a second electrode (either the opposite or same polarity). Alternatively, one medical component can include an ablation element or electrode while the second medical component contains one or more mapping electrodes to assess the ablation lesion created by the first medical component.

Variations of the system also include medical components also have a tether member or rail at a distal end thereof. The tether member can consist of a string or wire like structure that is used to simply pull the medical component through one or both arms. In additional variations, the tether member can include a flexible tether member that, when deflected, assumes a curvilinear shape based on the structural characteristics of the tether. As discussed below, this allows the medical component to assume a “U” shaped configuration that can assist in perforating the medical procedure.

The medical components for use in the system described herein can be configured to be advanceable over or relative to each other. In addition, the components can be configured to have mating portions that allow for releasable coupling the components together. In one variation, the ends of the components can include a male-female type connection such as those commonly used in medical device applications.

Additional variations of the medical components for use in systems described herein include a joint-member that couples components together, ideally, the joint member can either be a string-type member (having no column strength) or a flexible member that has a greater flexibility than either medical component. Possible additional variations include a coil, wire, spring, or similar structure. Such a configuration permits a physician to move the medical components closely together to form a “V” type configuration that may be useful in inserting the components in narrow passages or small openings.

In some cases, a physician might prefer use of a joint member during delivery of the system but not during performance of the medical procedure. In such cases, the system can be configured so that one or both medical components can negate the effects of the joint member. For example, a medical component can be configured to advance relative to a joint member. In this manner, a physician can simply uncover the joint member when desired and cover the joint member with the medical component when the flexibility provided by the joint member is not required. In a particular example, a device having a treatment component can be coupled to a tether via a joint member. However, the tether or treatment component can be advanceable over the joint member for respective coupling to negate the effects of the joint member.

Another aspect of the system is the ability to control movement of the distal portions of the arms from the proximal portions, in one variation, the arms will include handle portions on the proximal end. The handle portions can include one or more steering control mechanisms. The movement of the arms can occur in any three dimensional space. In some variations, it is important that the arms do not rotate, which reduces the chances of affecting a position of the medical component. Accordingly, any steering mechanism or steering control mechanism known by those skilled in the art is considered to be within the scope of this invention.

Turning now to the tether or flexible member: the tether or rail can be configured to have a circular, semicircular, oval, or non-uniform cross section. In additional variations, the flexible member or tether can comprise a guide-wire or guide tube with any number of openings for delivery of a fluid or substance.

In additional variations of the system, the medical components can be rendered lockable relative to one or both arms. In this manner, the arms can remain in a desired articulated position as one or more medical, components are positioned by movement of the arms.

In certain variations of the invention, one or more arms on any system can have a varying degree of flexibility along a length of the arm. For example, the arm can remain relatively stiff at a proximal portion and relatively flexible at a distal portion. This permits ease of articulation of the arms at the treatment site but a relatively stable proximal portion from which the arms are maneuvered.

In additional variations of the system, one or both arms can be configured to provide medical procedures as well. For instance, one or more arms can be configured to provide an electrode, a fluid source, a suction source, a reservoir to collect tissue, etc.

In an addition variation, the devices and method described herein include a medical system for performing a medical procedure on or in a patient, the system comprising a medical component, a first arm having a distal portion and a proximal portion, where manipulation of the first arm's proximal portion permits articulation of the first arm's distal portion and where the medical component is coupled to and advanceable relative to the distal portion, a second arm having a distal portion and a proximal portion, where manipulation of the second arm's proximal portion allows for articulation of the second arm's distal portion, a flexible member coupled to the medical component and extending through at least a portion of the second arm, and where the first and second arm are configured to be manipulated independently, and where manipulation of the first, or second arm alters a profile or position of the elongate section of the medical component allowing for positioning of the medical component.

Atrial fibrillation surgery is just one example of a surgical procedure that, while it relies on the surgical techniques discussed above, the procedure also suffers from shortcomings due to a lack of access to organs within the thoracic cavity. Atrial, fibrillation surgery involving radiofrequency, DC, microwave, ultrasound, laser or other modes of thermal ablation of atrial tissue has a limitation where tissue contact throughout the length of the electrode(s) is/are not consistent. Such inconsistent electrode contact causes variability in the transmission of energy throughout the target length of ablated/coagulated tissue. This inconsistency also produces undesirable gaps of viable tissue that promote propagation of wavelets that sustain atrial fibrillation, or produce atrial flutter, atrial tachycardia, or other arrhythmia. Target tissue regions that reside along the posterior surface of the heart is one factor that contributes to inconsistent electrode contact. As discussed above, conventional means of surgical access are not optimal to access the posterior surfaces.

In another variation, the delivery system comprises an elongate introducer, a deployable elongate guide member and a positioning element slidably and rotatably coupled to the guide member, the elongate guide member and positioning element slidably and rotatably positioned within the elongate introducer. The deployable elongate guide member has a longitudinal axis which defines a desired closed-loop operative path when deployed past the distal opening of the elongate introducer and within a patient's body. The positioning element is at least slidably coupled to the elongate guide member to define a position relative to the defined path from which a functional device is deployed.

In yet another variation, a delivery system is provided for delivering a functional device to a location within a patient's body, the delivery system comprising an elongate introducer, a deployable elongate guide member and a positioning element fixedly attached to, or otherwise integral to, the elongate guide member. The deployable elongate guide member defines an operative path when deployed past the elongate introducer and within a patient's body. The positioning element cooperates with the elongate guide member to define a position relative to the defined path from which a functional device is deployed.

In one aspect of the invention the functional device is deployable from the position along the defined operative path without requiring further engagement with the deployable guide system at a point along the elongate guide member distal to such position.

In another aspect of the invention the elongate guide member, as part of a guide system, includes a distal end which is positioned within the body when the elongate guide member is deployed. The single-ended elongate guide member may be linear, curvilinear or pre-shaped to address a specific desired operative path along the posterior wall of the heart between the left and right inferior pulmonary veins for example. In one aspect of the invention a positioning element slidably coupled to a preshaped single-ended elongate guide member deployed within a hollow organ cooperates with the deployed guide member to access a majority of the tissue surface of the hollow organ without redeployment of the guide member.

In yet another aspect of the invention the elongate guide member, as part of a guide system, is adapted to define or form a loop-shape when deployed. The elongate member may have a rectangular cross-sectional geometry to encourage deflection in a single plane. In contrast, the elongate member may have a circular cross-sectional geometry allowing the loop-shaped guide member to be directionally positioned, or otherwise steerable, further allowing a user greater flexibility in defining the operative path.

The devices described herein can be fabricated from known materials that are commonly used for medical device applications. For example, PBAX, PTFE, or other polymeric materials can be used for the body portions of the medical component, arms, or handles. In addition, stainless steel, Nitenol, suture thread, mono-filament (e.g., fishing line-type of materials), suture thread, other materials can be used as the tether or flexible joint as described herein. In some variations, a Drawn Filled Tube (DFT) such as those provided by Fort Wayne Metals, Fort Wayne, Ind., can be used for the tether. The DFT material comprises a first material or shell over a second material having properties different from the outer shell. For example, a DFT wire can comprise a superelastic (e.g., Nitinol) outer tube with a radiopaque material within the super-elastic outer shell. This material provides shape-memory alloy properties as well as radiopaque attributes.

The invention also includes methods of performing the acts of deploying, positioning and treating tissue using the systems described herein. In one example, the method includes performing a procedure on tissue, by positioning at least a first and second arm adjacent to the tissue, where each arm includes a distal portion and a proximal portion, where manipulation of each arm's proximal portion causes movement of the respective arm's distal portion independently of the other arm, advancing a medical component through the first arm to the tissue such that a portion of the medical component is coupled to the second arm, manipulating either the first or second arm within a three dimensional area and independently of the other arm to alter a profile or location of the medical component; and performing the procedure with the medical component.

### BRIEF DESCRIPTION OF THE DRAWINGS

The following figures provide examples of various configurations of the invention as described herein. The invention includes combinations, where possible, of the different aspects of embodiments, or even combination of the embodiments themselves.

FIG. 1 illustrates a variation of an inventive medical system allowing for improved positioning of one or more medical component.

FIG. 2A represents an example of an arm that can be steered or maneuvered through articulation of a distal end and/or rotation of the arm.

FIG. 2B is a cross sectional view of the arm of FIG. 2A where the arm has a single steering wire or mechanism.

FIG. 2C shows an example of an arm that can be steered or maneuvered through a three dimensional space without rotation of the arm.

FIG. 2D is a cross sectional view of the arm of FIG. 2C showing the arm to have a plurality of steering wires or mechanisms.

FIG. 3A shows an example of a system where a first and second component can be positioned relative to one another. FIG. 3A also illustrates use of a third steering arm to assist in positioning either component.

FIG. 3B shows an alternate variation of a system of the present invention where one or more medical components are coupled to the arms via coupling sections that allow advancement of the components adjacent to the arms rather than coaxially.

FIG. 4 is an example of a system of the present invention being advanced through the body and into a heart of a patient.

FIGS. 5A-5C show an example of a system having a single component extending between a pair of steerable arms in a loop configuration and being manipulated to a desired profile.

FIGS. 5D-5E show an example of a single device having multiple components as the device is positioned in a three dimensional profile.

FIG. 6A shows a flexible tether coupled to a medical component where the tether has sufficient rigidity to assume a curve or profile.

FIG. 6B shows a “floppy” or “string-like” tether coupled to a medical component and extending to a second arm.

FIG. 6C shows a second component, being advanced over a flexible tether such that the first and second components can be removably coupled.

FIG. 6D shows the system of FIG. 6B where the first and second components can be removably coupled to function as a unitary device where flexure of the tether or medical components causes the system to assume a “U” shaped configuration.

FIG. 6E shows a variation of a system where a single device can be advanced between a pair of arms.

FIG. 7A illustrates a system where a first medical component comprises a first medical device is coupled to a flexible tether via a joint member.

FIG. 7B illustrates the variation of FIG. 7A where the joint member allows for the system to assume a “V” shaped configuration.

FIG. 7C illustrates another variation in which two separate medical components are joined by a flexible joint without a tether or flexible member.

### DETAILED DESCRIPTION OF THE INVENTION

Generally, the devices and methods described herein provide a physician with an improved ability to remotely position one or more medical components on or in a target tissue location. The ability to position such medical components allows the physician to perform one or more medical procedures with improved accuracy and efficiency where direct use of the physician's arms is not possible. Although variations of the system described below primarily discuss placement of one or more energy delivery devices on tissue, such variations are for exemplary purposes only. The features of the system can be used to position any number of additional medical components (including implant delivery devices, diagnostic devices, imaging devices, biopsy devices, radiation emitting devices, drug delivery devices, radio-opaque markers, valvular annuloplasty devices, suturing devices, implants, tissue remodeling devices, fluid delivery catheters, introducer devices, electrosurgical devices adapted to deliver a variety of energy modalities, needles, injecting devices, chemical eluting devices, etc.) The system could also be used as a stable platform to provide minimally invasive surgical procedures. In such procedures, the system could be used to cut, suture, coagulate or remove tissue for example.

In addition, any suitable imaging modality may be used to visualize the anatomy and/or one or more of the devices disclosed herein while performing a procedure. Examples of such imaging modalities include, but are not limited to endoscopes (e.g. colonoscopes, laparoscopes, thoracoscopes, bronchoscopes, cystoscopes, colposcopes, hysteroscopes, arthroscopes, etc). X-rays, Computed tomography (CT), fluoroscopy, ultrasound, MRI, PET, near infra-red imaging, etc.

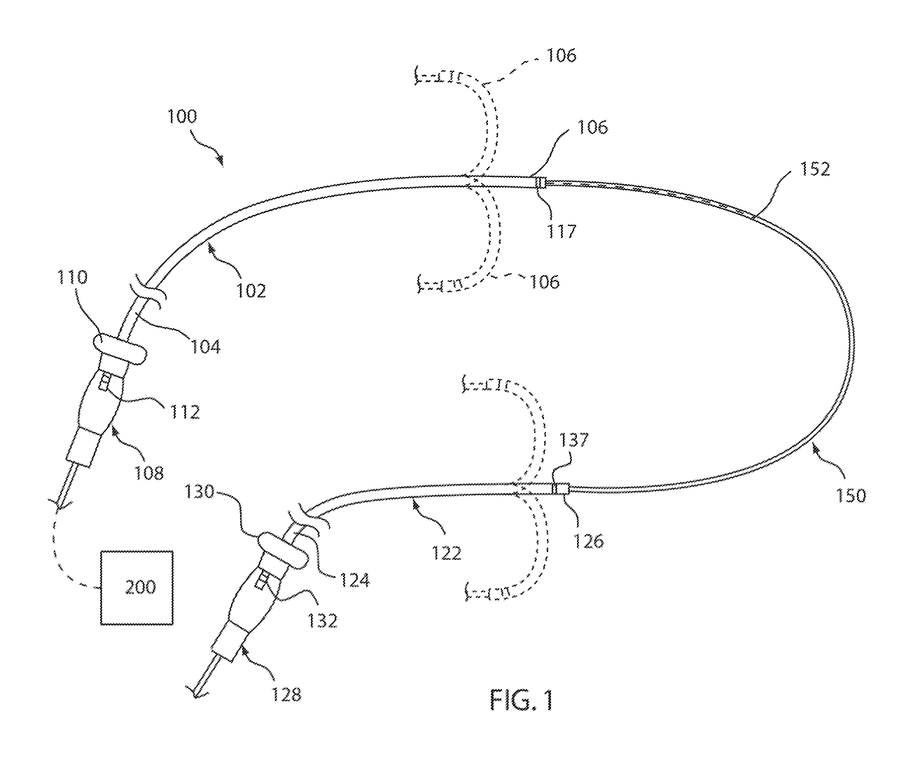

FIG. 1 illustrates a first variation of a medical system 100 that allows for improved positioning of one or more medical components 150. As shown, the system 100 includes a first 102 and second 122 maneuverable or steerable arms where adjustment of the arms at a proximal portion 104 124 positions the medical component 150 extending between the distal portions 106 126 of arms 102 122. As discussed in detail below, the system 100 can be equipped to position any number of medical components. In most cases, at least one medical component 150 shall extend, between arms 102 122. In additional variations, the first medical component 150 can be used to provide a path for a second medical component (not shown) that performs the medical procedure. For example, a variation of the device can include the first, medical component 150 comprising a rail or guide-wire type of device. Once the physician positions the rail guidewire, the physician can then advance a second treatment or therapeutic component along the rail/guidewire to perform a diagnostic or treatment procedure. In an alternate variation, the second treatment component can be a catheter with a working element (where such working element can comprise an electrode for mapping and/or ablation, a sensor, or other active portion), in yet another variation, the first maneuverable or steerable arm 102 can be non-movably coupled to the medical component 150. In such a case, the combination of the first permanently mounted steerable arm and the medical component would itself become a first maneuverable or steerable medical device. The medical device could include working elements, where such working elements can comprise an electrode for mapping and/or ablation, a sensor, or other active portion. The medical device could also be passive with no working element. Examples of a medical device with at least one working element are any steerable ablation catheters using ablative energies such as radiofrequency, microwave, ultrasound, cryo, or laser. Similarly, both the first and the second maneuverable or steerable arms 102 and 122 could non-movably coupled to the medical component 150. In such a case, the combination of the first and second permanently mounted steerable arm and the medical component would itself become a first maneuverable or steerable medical device. The medical device could also include working elements as described earlier in this paragraph, or include no working element and be passive.

In additional variations, the medical component ISO itself can comprise an active element (not shown) such that it performs a diagnostic or treatment procedure. In those cases where the medical component 150 requires connection to an auxiliary component (e.g., a power supply, imaging monitor, fluid source, etc.) the medical component 150 can extend through either handle 108 128 for connection to a respective auxiliary unit 200. In some variations, the handle can include the desired connector. One or more regions of medical component 150 can be positioned at a desired location and orientation by advancing or withdrawing one or both arms 102 122; torquing one or both arms 102 122; deflecting or maneuvering or steering the distal portions 106 126 of arms 102 122; and advancing or withdrawing medical component 150 relative to the distal ends of arms 102 122. In yet an additional variation, the medical component 150 can comprise a hollow rail (as illustrated in FIG. 1), where a medical treatment component 152 can be slidably located therethrough. For example, the medical treatment component 152 can consist of an energy transfer device that delivers energy through the rail (or an opening located therein) to perform the surgical procedure.

In additional variations, the arms 102 122 themselves can be designed to provide any number of medical procedures. For instance, one or both the arms 102 122 can have an electrode or active element located on an exterior. In another example, the distal portion 106 126 of the arms 102 122 can be designed to provide suction, irrigation, contrast agents, etc. to the target site, in yet another variation, the distal portion 106 126 of the arms 102 122 can be fitted with vision capabilities such as a fiber optic, CCD camera, or another vision source enabling direct visualization of the medical component 150 during the procedure.

As will be apparent throughout this disclosure, any number of system 100 permutations is possible. In some variations, the system 100 is configured so that both of the arms 102 122 are structurally similar. However, systems 100 under the present disclosure can include a first and second arm 102 122 having different structural features (such as the degree to which the arm can be steered, the diameter, flexibility, etc.). For illustrative purposes only, both arms in FIG. 1 are shown to have the same structural features. Generally the arms include a proximal portion 104 124 and maneuverable distal portions 106 126. The arms 102, 122 permit steering or repositioning of the maneuverable distal section 106 126 (as shown by the dashed lines of FIG. 1) from the proximal portion 104 124 of the arm 102 122. This allows a physician to remotely control the position and/or the orientation of the distal opening of the arm. Typically, the system 100 includes the use of handle member 108 128 located at the proximal portion 104 124 of the arms. However, the arm 102 may be configured to operate without a handle. In such a case, the proximal portion 104 would couple to an automated or computer controller assembly.

The first and second arm 102 122 may be introduced into an anatomical region through natural or artificial openings to that anatomical region, in such a case, the first and second arm 102 122 may be introduced through, the same opening or through, two different openings. In one variation of a method under the present invention, first and second arm 102 122 are introduced into the left atrium of the heart through a single trans-septal opening from the right atrium of the heart. In another variation, first and second arm 102 122 are introduced into the left atrium of the heart through two trans-septal openings from the right atrium of the heart.

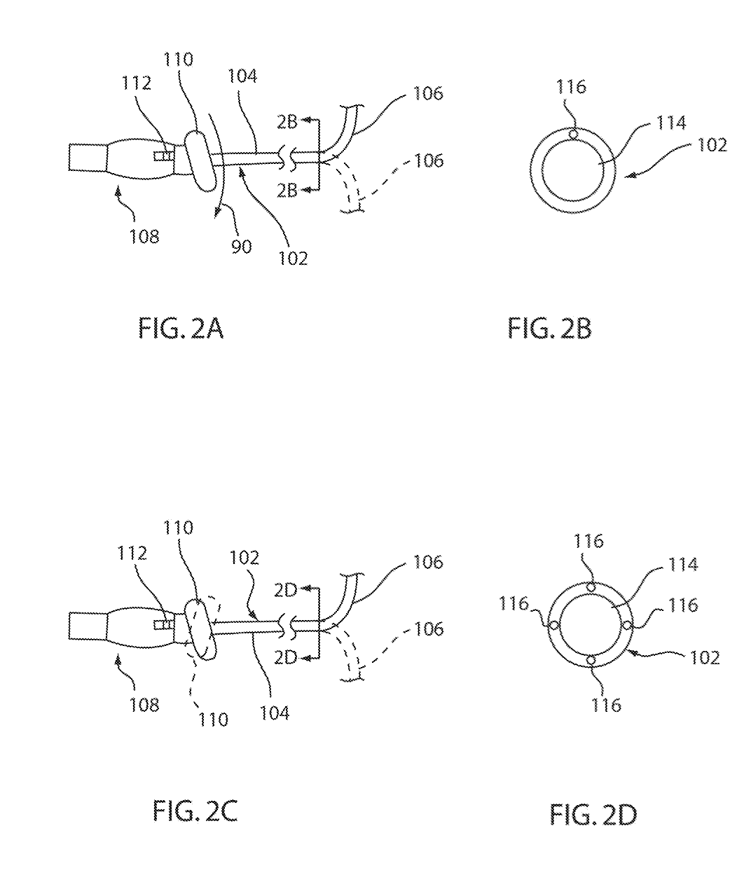

FIGS. 2A and 2C illustrate variations of steerable arms 102. In this variation, the arm 102 includes a proximal portion 104 having a handle 108. The handle 108 includes at least one steering mechanism 110 that allows the physician to control the maneuverable distal section 104 with a single hand. Accordingly, the physician can manipulate the maneuverable distal section 126 of the second arm 122 by maneuvering the second handle 128 with his/her other hand. The arm 102 can be configured to use any known steering assembly such as pull wires, pre-shaped tubular sheaths or stylet structures, or any other known steering mechanism as known to those skilled in the field of steerable medical components. However, FIGS. 2A and 2C illustrate two exemplary variations for positioning the distal section 106 of the arm 102.

FIG. 2A shows a first variation where deflection of the arm 102 occurs as the physician manipulates the steering mechanism 110 to produce a desired degree of articulation. To reposition the distal section 106, the physician can then rotate the arm 102 as shown by arrow 90. This causes the articulated distal section 106 to move in an arc-type motion (e.g., the dashed lines in FIG. 2A). FIG. 2B shows a cross section of the steerable arm 102 of FIG. 2A taken along the lines 2B-2B. As shown, the steering mechanism 110 can be coupled to a single steering wire or member 116 that extends through a wall of the steering assembly so that the wire does not interfere with any devices located in the arm's 102 working lumen.

In some cases it will be desirable to reposition the arm 102 along a three-dimensional an arc-type path, space, or area without requiring rotation of the arm 102 or handle 108. Where rotation of the arm might also rotate the medical component located therein, the ability to reposition the arm without rotation may be useful to avoid or minimize rotation of at least a proximal portion of the medical component. As shown, movement of the steering mechanism in a first position produces movement of the distal portion 106 as shown. As the physician moves the steering mechanism 110, the distal portion 106 repositions without rotating (as illustrated by the dashed lines in FIG. 2C). Although there are many variations capable of producing this type of non-rotational steering. FIG. 2C illustrates one possible variation. FIG. 2D illustrates a cross sectional view of a steering arm 102 of FIG. 2C taken along line 2D-2D. In this example, the steering arm 102 includes a plurality of steering members 116 arranged in a wall of the arm 102. Although the illustrated variation shows four (4) steering members 116 devices according to the present invention can include any number of steering members as desired without adversely impacting the size or flexibility of the arm 102.

As also noted above, the handles 108, 128 can include optional locking mechanisms 112, 132 that permit the physician to independently lock each arm 102 122 into a desired profile or orientation thereby reducing the need of the physician to maintain continuous exertion to hold the arm in any particular profile. It should be noted that any number of steering mechanisms 110 can be employed in the systems described herein.

For purposes of illustration, a single mechanism is shown on each arm. However, the invention is not limited to that shown. Instead, the arms of the present invention may employ any number of controls or actuators to produce the desired steering capability of the device. In addition, devices may be configured to have different steering capabilities based upon the intended target region. For example, devices used in endovascular cardiac applications might employ different steering capabilities than devices used thoracic cardiac applications. In addition, certain variations of the system include arms that are moveable, steerable, or positionable, through a three dimensional space. However, the system may include variations where one or both arms are moveable, steerable, or positionable in a single plane.

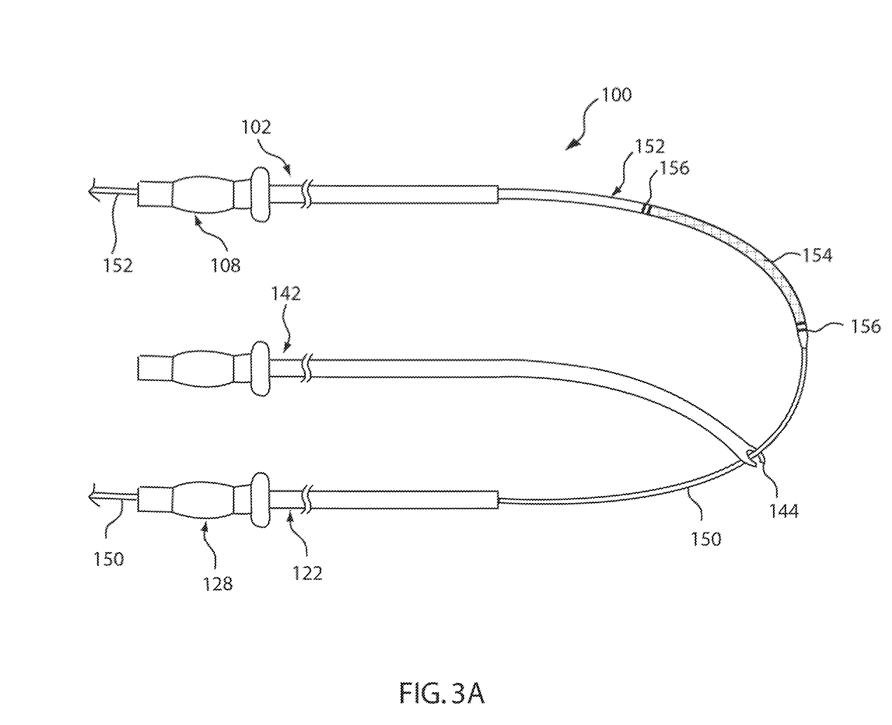

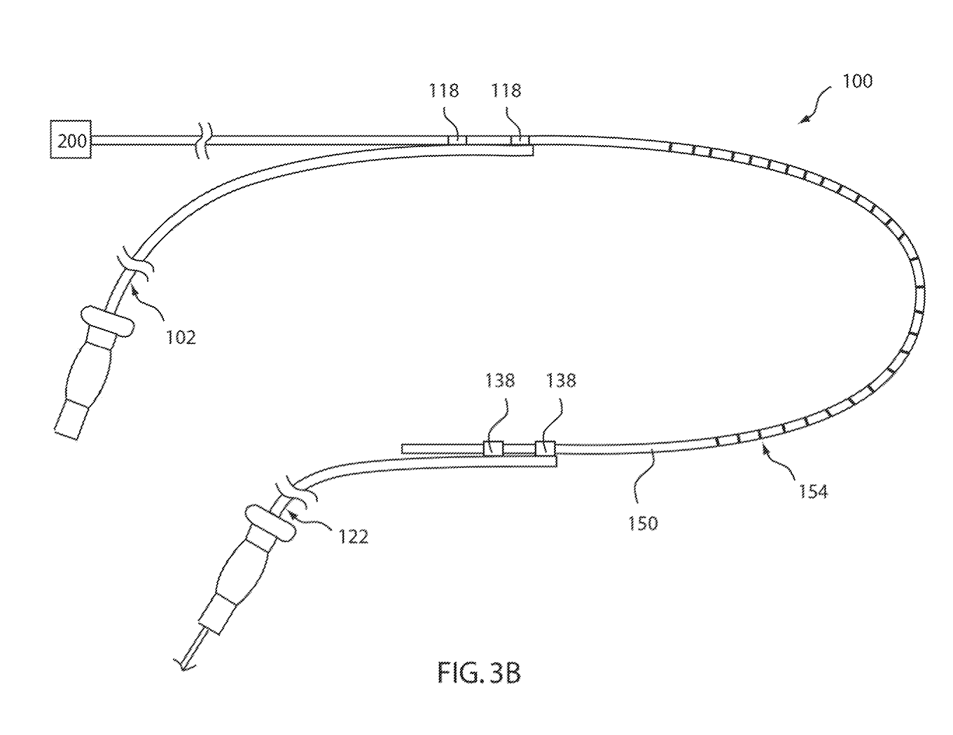

FIG. 3A shows another example of a system 100 where a second medical component 152 advances over a first medical component 150. In the illustrated variation the second medical component 152 comprises an energy delivery catheter and the first component comprises 150 comprises a tether or rail. As noted herein, the system 100 can be used with any number of medical components. In the illustrated variation, the second medical component 152 comprises an energy transfer device having an active region 154 along with one or more additional energy transfer elements 156. The energy transfer elements 156 discussed herein can include any variety of transfer elements. For example, the energy transfer element can comprise a one or more radiofrequency electrodes (monopolar or bipolar), a DC resistive heating source, a microwave antenna, ultrasound energy transfer element, laser source, source of cryogenic energy. Alternatively, or in combination, one or more energy transfer element can also comprise sensor or information receiving elements. For example, such elements can include electrodes (that detect voltage, current and/or impedance), temperature sensors, pressure sensors, Sight measurement devices, infrared sensors, chemical sensors, radiation sensors, deformation sensors, or any other type of sensors that observe or measure a state or condition of tissue or the body.

As shown, the second medical component 152 can advance over the first medical component 150 which functions as a rail, guide, or tether. In such a case, the first and second medical components 150 and 152 comprise separate medical devices. In an alternative variation, the first and second medical components 150 and 152 can be integrated in a single device where the energy transfer device or component 152 includes a rail or tether that is affixed to a distal end. In such a case, rail or tether can be used to either pull the device through the second arm 122 (as a tether member) or provide sufficient column strength and flexibility to aid in positioning of the active region 154 as desired (as a flexible member or flexible tether). In another variation of the system 100, the first, and second medical components 150 and 152 can be integrated together as a single device and a portion of the second component 152 is torqueable and the rail or tether portion of component 152 is non-torqueable. Such a variation can be designed, for example, by having the rail or tether portion be more flexible than the torqueable portion of component 152. Thus when the torqueable portion is twisted by a user, the twist is substantially transmitted throughout the torqueable portion and if the rail or tether portion is twisted, the twist is not transmitted through the rail or tether portion.

FIG. 3A also illustrates the use of an optional third arm 142 that is coupled to either medical component 150 or 152. In the illustrated variation, the third arm 142 comprises a grasping structure 144 used to removably engage either medical component 150 or 152. The third arm 142 allows a physician with another means of positioning the medical components to perform the desired procedure. The grasping structure 144 allows the third arm 142 to be introduced into or removed from the operative space at any point during the procedure. Any number of grasping mechanisms can be employed including hooks, rings, grasping jaws, etc.

FIG. 3B illustrates a variation of a system 100 under the present invention where the medical component 150 is coupled adjacent to the arms 102 and 122 via coupling portions 118 138 rather than being coupled through the arms as shown in the previous figures. As noted above, the coupling portions 118 138 can comprise ring or tube structures. Alternatively, the coupling portions 118 138 can consist of the grasping mechanism discussed above. In this variation, the medical component 150 itself comprises an energy transfer region 154.

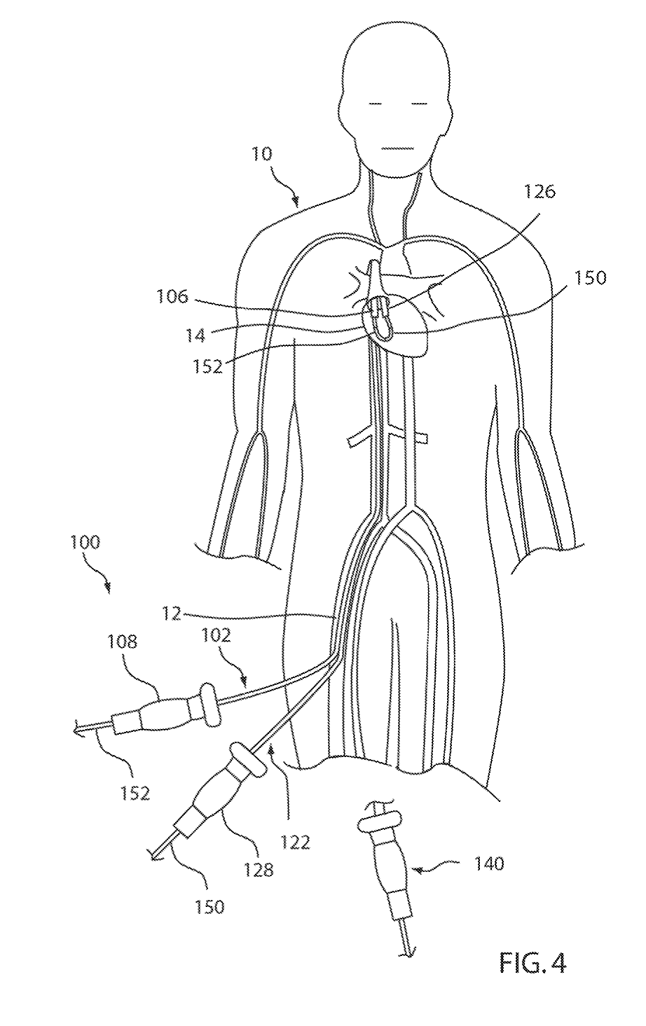

FIG. 4 illustrates one possible use of a system 100 according to the present invention. In this example, the system is used to perform an endovascular cardiac procedure using one or more medical components 150 152. As shown, a physician directs first and second arms 102 122 into the body 10. In this variation, the first and second arms 102 122 are inserted through the femoral vein 12 and ultimately advanced into a right atrium of the heart 14 via the interior vena cava. Ultimately, the system 100 can be advanced into any chambers of the heart (for example a transeptal crossing technique could be used to access the left atrium to produce complex ablation patterns such as the Maze lesion pattern). However, any known access methods may be used to gain entry.

As noted above, the arms 102 122 can be coupled via first and/or second medical components 150 152 when inserted into the body. Alternatively, the system 100 is configured so that the medical component's) 150 152 can be advanced in and out of (or distal and proximal to) the arms 102 122. This construction allows a physician to remove the medical component 150 from the first 102 or second arm 122 to ease insertion of the respective arms 102 122 into the patient's body. For example, the arms can be inserted into separate entry point or each arm can be separately maneuvered to the target site where the arms are ultimately coupled. In other words, the system 100 can be inserted into a patient without the arms 102 122 being coupled by the medical component 150. Once the arms 102 122 are positioned at or near the target site; the medical component 150 can be advanced, from one steerable arm to the other arm. In some variations, the medical component 150 can be affixed to one arm so that it engages that, arm without extending through the entire length of the arm. Alternatively, the medical component 150 can be advanced from one arm, through the respective other arm until the device extends out of that arm's handle member 108 128 as shown in FIG. 4.

FIG. 4 also shows a third steerable arm 140. As noted above, the system 100 can include any number of additional steerable arms that can be used to aid in positioning the medical component 150. In any case, once the physician places the system 100 within the target site (e.g. a chamber of the heart or other endocardial tissue), the arms 102 122 provide the physician with the improved ability to maneuver the medical component or components 150 152 using the steerable distal portions 106 126 of the arms 102 122.

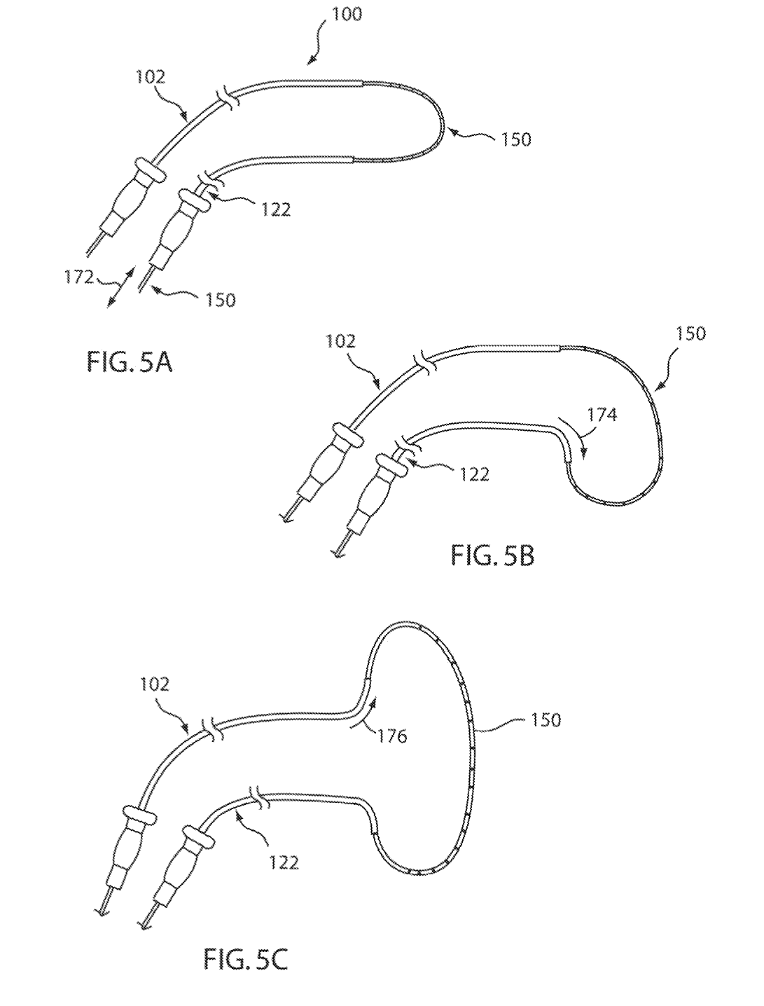

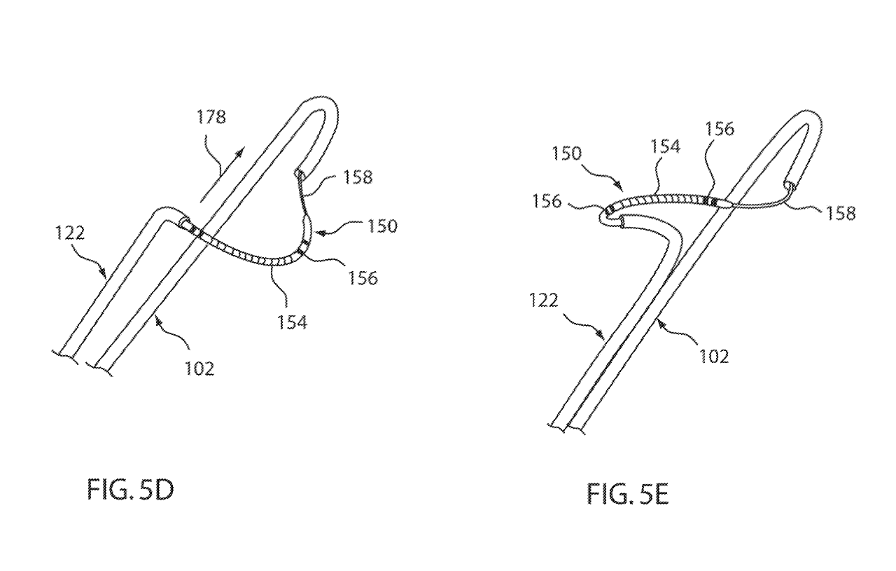

FIG. 5A illustrates another example of a system 100 where a medical component 150 extends between arms 102 122 forming a loop or (or other three dimensional profile). As shown by arrow 172, the medical component 150 can be advanced or withdrawn from either arm 102 122 to adjust a size of the loop formed by the medical component 150.

Next, either arm 102 122 can be articulated, steered, or maneuvered to adjust a profile of the medical component 150. In this variation, the second arm 122 is repositioned, as shown by arrow 174 of FIG. 5B, to adjust the profile of the medical component 150. The independence of arms 102 122 allows adjusting of the profile of the medical component 150 without substantially disturbing the state and location of the first arm 102.

Finally, if desired, the physician can adjust the respective other arm (in this example, arm 102—as illustrated by arrow 176 of FIG. 5C) to position the medical component 150 in a desired profile. Upon satisfactory placement, the physician can carry out the intended medical procedure.

Although the examples illustrated above depict movement of the medical component 150 in two dimensions, the system 100 provides many degrees of freedom to position the medical component 150 in any number of positions or profiles from simple curves to complex three dimensional profiles that conform to a targeted region, of tissue. For example, as shown in FIG. 5D, the arms 102 122 can be advanced relative to one another and then articulated to orient the medical component 150 in a first profile. Subsequent articulation of the second arm 122 allows a physician to place the medical component 150 as desired against a contoured surface of tissue as shown in FIG. 5E. FIGS. 5D and 5E also illustrate a variation where the medical component 150 comprises an energy delivery catheter having an active region 154 and a number of additional electrodes 156. The medical component 150 also comprises an integral tether 158.

In another variation, the proximal portion of the non-tether region of component 150 encloses a coaxial cable for delivering microwave energy to the active region 154. The coaxial cable comprises an inner conductor, an outer conductor and a layer of dielectric between the inner conductor and the outer conductor. As one example of such a device, the inner conductor comprised a silver plated stainless steel with a diameter of 0.46 mm. The dielectric comprised expanded PTFE with an outer diameter of 1.12 mm. The outer conductor has an outer diameter of 1.40 mm. The active region 154 encloses a microwave ablation antenna. The antenna is formed by removing a length of the outer conductor and the dielectric to expose the inner conductor. The distal end of the microwave antenna may be electrically connected to a metallic cap. A layer of silicone may be added around the microwave antenna to maintain a uniform cross sectional profile along the length of the component 150. The coaxial cable and/or the microwave antenna may be covered in a layer of PTFE having an outer diameter of 1.65 mm. Clearly any additional variations of devices are within the scope of this disclosure.

The additional electrodes in this above variations may be electrophysiological mapping electrodes. The conductors leading to such electrodes may be arranged to minimize the distortion of a microwave field generated by the microwave antenna. In the variation shown in FIGS. 5D and 5B, the catheter 150 comprises an integral tether 158. In an alternate variation, the distal end of the medical component 150 is reversibly attached to tether 158. In this variation, the distal end of the medical component 150 comprises an opening. The opening functions essentially like the eye of a needle allowing a user to introduce a looped tether 158 through the opening. In this variation, the tether 158 may be made of suitable metallic or non metallic materials.

In another variation the tether 158 is made of a radiopaque metallic material or a material treated to have radiopaque attributes (such as the “DFT” material described above). An advantage of this construction is the ability to disconnect the tether 158 from the medical component 150 remotely by opening the tether 158 loop at a remote location (e.g. at a proximal region of the tether 158) and pulling a free end of the tether 158.

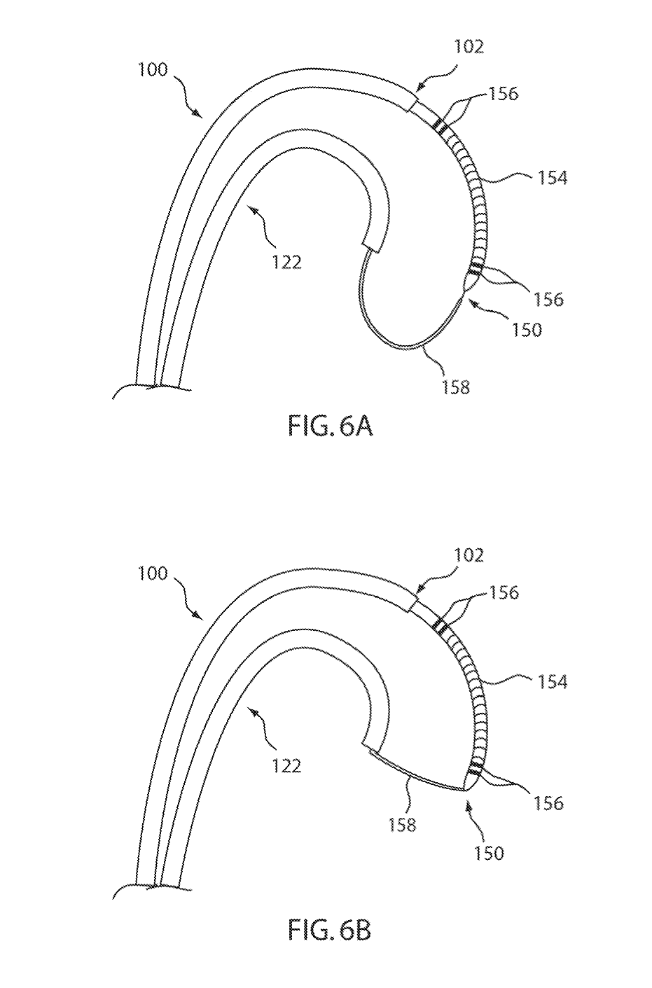

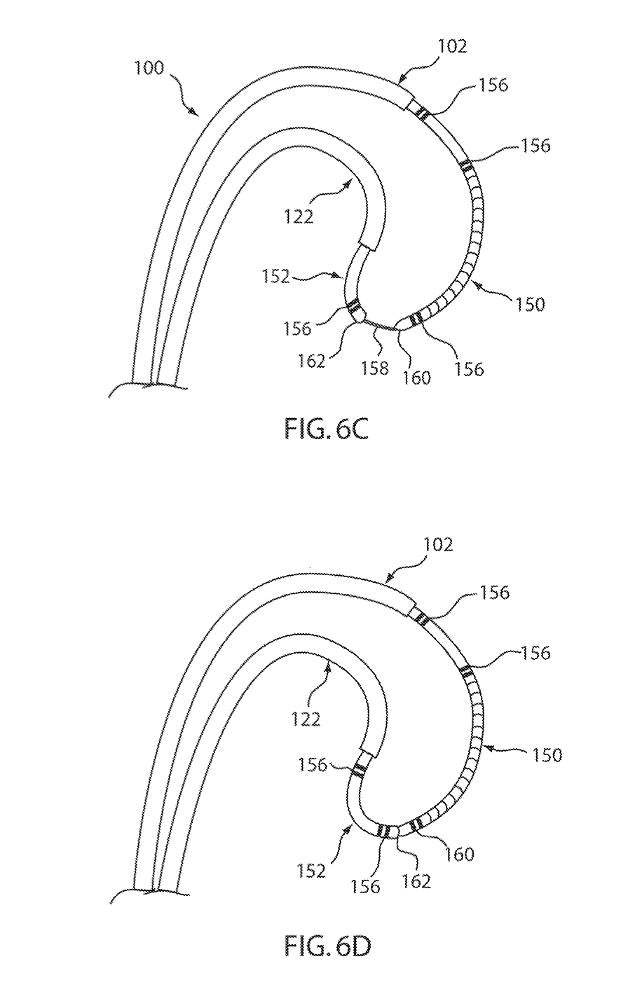

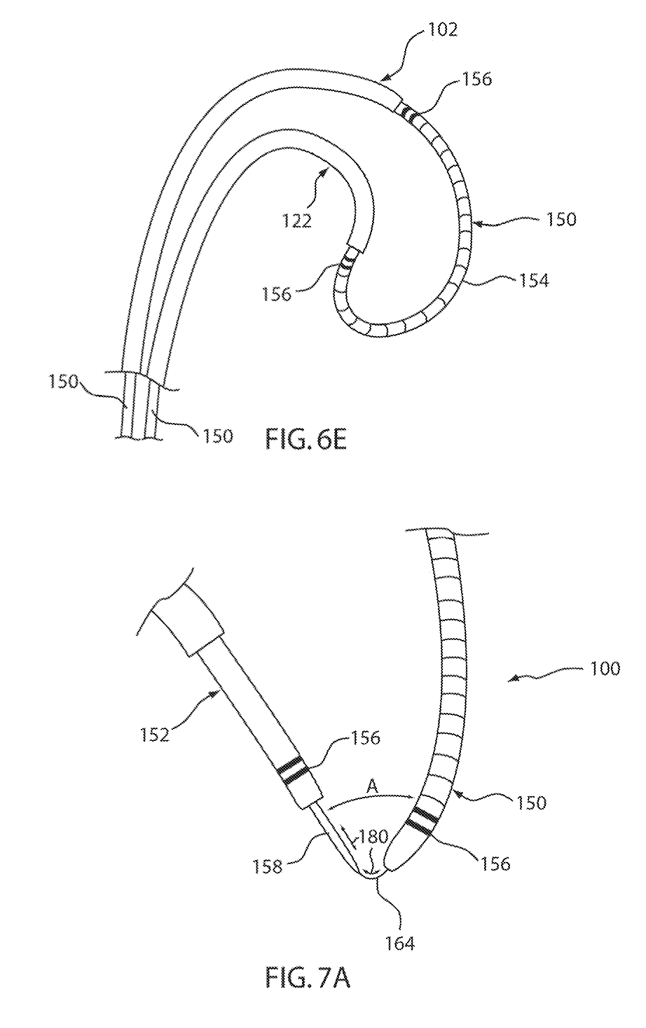

While some variations of the system allow for the tether that has little column strength (essentially used to pull the device), alternate variations of the tether include a wire-like member having a sufficient flexibility that allows for improved control of an end of the catheter that is coupled to one arm 102 while the other arm 122 is repositioned. FIG. 6A illustrates this configuration as the energy delivery device 150 includes an integral flexible tether 158 extending from a distal end and advanceable into a second arm 122.

As shown in FIG. 6A, the tether can comprise a flexible member that when bent assumes a minimum bend radius. Accordingly, such a tether 158 can be fabricated from a metal alloy (e.g., stainless steel, Nitenol, DFT, etc.). The tether can also be fabricated from a polymeric material having resilient properties, in any case, the use of a flexible tether 158 allows manipulation of the tether to assume a profile for guiding one or more of the medical component. In contrast, an alternate variation of the system 100 includes a tether 158 as shown in FIG. 6B. In this variation, the tether is floppy or thread-like. This allows for direct positioning of the medical component 150 but without the tether assuming a non-linear shape. Accordingly, any number of materials may be used, including but not limited to stainless steel, Nitenol, suture thread, mono-filament (e.g., fishing line-type of materials), suture thread, other materials so long as the tether is floppy.

FIG. 6C illustrates a second variation of a working end of a system 100 according to the present invention. In this variation, a second medical component 152 comprises a catheter 152 that is advanced over the tether 158 of the energy delivery device 150. The second medical component 152 can consist of any number of medical devices as described herein. In the illustrated variation, the second medical component 152 comprises a catheter tube having mapping electrodes 156. As noted above, the catheter tube 152 can be coupled to any number of auxiliary sources to deliver fluid (such as saline or a contrast agent), provide suction, deliver additional medical devices/components, or provide visualization.

FIG. 6C also illustrates an example of the medical components 150 152 having respective mating portions 160 162. When the medical components 150 152 are advanced together, the mating portions 160 162 couple together allowing for the device to behave as a single unitary device. The medical components 150 152 and the mating portions 160 162 maybe be designed to ensure a smooth transition of the outer surface of medical components 150 152 when the mating portions 160 162 couple together. In one variation, mating portion 160 comprises a tapering region and mating portion 162 comprises a hollow region. As noted above, the medical component 150 for use with the systems described herein can be energy delivery devices. In the illustrated variation, the medical component 150 comprises a microwave ablation device with electrodes 156. In an additional variation, the cross-sectional profile of the tether 158 is smaller than the cross-sectional profile of the rest of the medical component 150. Thus the loop formed in the target anatomy by the medical component 150 beyond the distal ends of arms 102 and 122 is asymmetric—having a slimmer tether 158 and a thicker rest, of the medical component 150. In this variation, second medical component 152 comprises a hollow region adapted to slide over tether 158. When the medical components 150 152 are advanced together with a force, the mating portions 160 162 couple together allowing for the device behave as a single unitary device.

As shown in FIG. 6D, the coupled device can advance between adjacent arms 102 122 in a similar manner as a single unitary device. Naturally, additional variations of the systems described herein can include a single device 150 extended between adjacent arms 102 122 (as shown in FIG. 6E). In such a case, the single device 150 can extend fully through the arms 102 122 and exit at the distal proximal portions of the arms 102 122 to allow for manipulation of the device and at the same time the device 150 can extend between arms (as shown in FIG. 6E) for performing the medical procedure). Alternatively, one or more ends of the device 150 can terminate in the arms 102 122 so that the device does not need to extend through the entirety of both arms. As shown, in those variations where the device includes a tether, the flexible nature of the tether 158 results in a curved profile that provides a degree of opposition force to the distal portion of the energy delivery device 150. This permits the device 150 to maintain a “U” configuration or shaped profile between the arms 102 122. However, even in those variations not including a tether, the flexible nature of the device 150 or devices 150, 152 allows for the formation of the “U” configuration.

In additional variations of the system 100, the single device 150 of FIG. 6E comprises one or more working elements. Examples of such working elements include, but are not limited to diagnostic electrodes (e.g. mapping electrodes), ablating elements (radiofrequency ablation electrodes, microwave ablation electrodes, cryogenic ablation elements, laser ablation elements, thermal ablation elements, high intensity focused ultrasound ablation elements, etc.)

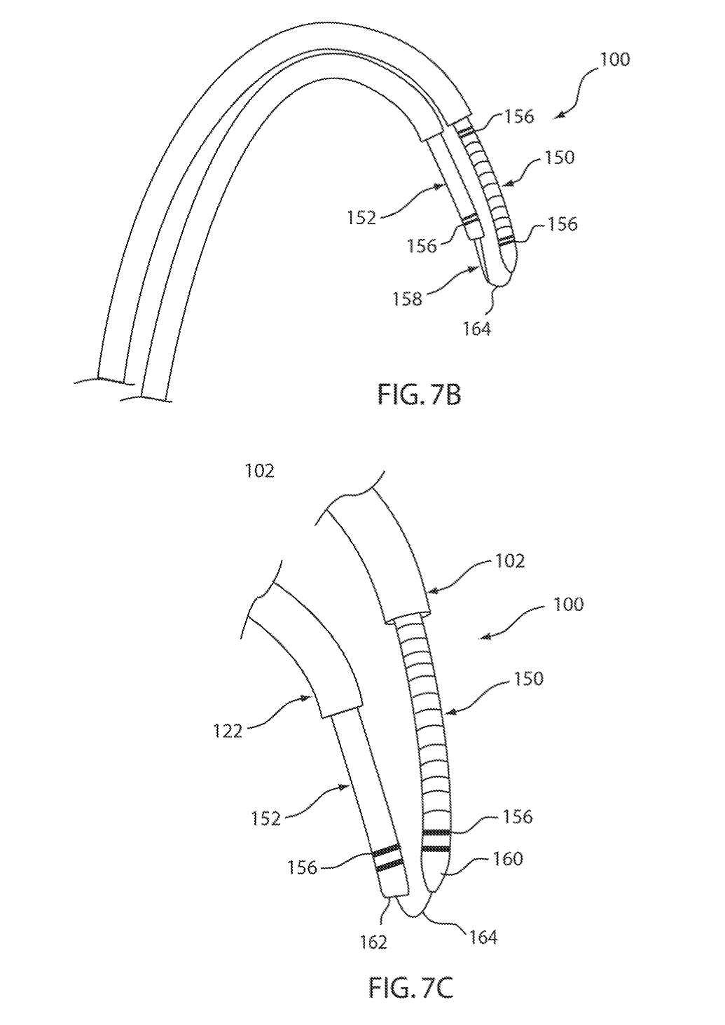

FIG. 7A illustrates a system 100 configuration where a first, medical component, comprises a first medical device 150 having a flexible tether 158 and also including a joint member 164 coupling the tether 158 to the energy delivery device 150. Generally, the joint member allows for increased articulation between the flexible tether 158 and the medical device 150. This permits the medical device 150 and tether 158 to form a smaller angle A than would not otherwise be attainable given a flexible tether 158 (as illustrated in FIG. 6A). Basically, such a “V” configuration allows for the medical device 150 and tether 158 (or other component that is joined to the medical device) to be inserted into a smaller opening or passage, in some cases, the components joined by the joint member 164 can be placed substantially parallel to each other (as shown in FIG. 7B). The joint member 164 can be a flexible joint or it can simply be a “string-like” structure that allows for the joined medical components to be connected but easily positioned into the “V” configuration.

In some circumstances, a physician needs the ability to selectively use a joint member. For example, a joint member may be useful when advancing the medical components to a target site using a narrow profile (as shown in FIG. 7B), but not desired when the components are positioned at the target site (e.g., the profile shown in FIG. 6A). Accordingly, some variations of the system 100 one or more of the components joined to a joint member are moveable relative to the joint member. For instance, in referring to FIG. 7A, the tether 158 can be axially moveable relative to the joint member 164 as shown by arrows 180. Therefore, the medical device 150 and tether 158 can be de-coupled to assume the “V” configuration (as shown in FIG. 7B) during advancement to the target area. Upon reaching the target site, the medical device 150 or tether 158 can be moved relative to the joint 164 and directly coupled to assume the “U” configuration shown above.

FIG. 7C illustrates a system 100 configuration where a first medical component or device 150 is detachably coupled to a second medical component or device 152 directly through a joint member 164 but without any rail or tether. As illustrated, the devices 150 152 can include coupling portions 160 162 so that upon joining, the joint member 164 is rendered ineffective.

The systems and methods described above are applicable to the delivery of various medical components within or on a body for performance of one or more medical procedures. The system provides a physician or other medical practitioner with an improved ability to manipulate and position the medical components, accurately and reliably, for the performance the medical procedures. The present invention is not limited to the examples described above. Various modifications, equivalent processes, as well as numerous structures to which the present invention may be applicable will be readily apparent to those of skill in the art to which the present invention is directed, upon review of the present specification.

The above illustrations are examples of the invention described herein. It is contemplated that combinations of aspects of specific embodiments or combinations of the specific embodiments themselves are within the scope of this disclosure.

We claim:

1. A medical system for performing a medical procedure on or in a patient, tire system comprising:
at least a first and second arm, each arm having a maneuverable distal portion and a proximal portion, where manipulation of the respective arm's proximal portion permits articulation of the arm's maneuverable distal portion independently of the other respective arm; and
a first medical component coupled to the first arm and extendable from the first arm's maneuverable distal portion, the first medical component being engageable with the second arm's maneuverable distal portion, such that when coupled to both arms, movement of either maneuverable distal portion alters a profile or position of the first medical component allowing for positioning of the first medical component.

2. The medical system of claim 1, where the first medical component further comprises a tether member at a distal end thereof.

3. The medical system of claim 2, where the tether member further extends through the second arm's maneuverable distal portion to the proximal portion.

4. The medical system of claim 1, where the first arm comprises at least one electrode on an outer surface.

5. The medical system of claim 4, where the second arm comprises at least one electrode on an outer surface.

6. The medical system of claim 1, further comprising a second medical component coupled to the first medical component and extendable through the second arm's maneuverable distal portion.

7. The medical system of claim 6, where the second medical component comprises at least one electrode.

8. The medical system of claim 6, where the second medical component includes a second lumen extending therethrough, and where the second medical component is advanceable over a portion of the first medical component.

9. The medical system of claim 8, where the second medical component comprises a fluid delivery catheter.

10. The medical system of claim 6, where the first medical component comprises a first mating portion and where the second medical component comprises a second mating portion, where the first and second mating portions releasably couple to each other.

11. The medical system of claim 6, where the first medical component and second medical component are detachably coupled.

12. The medical system of claim 6, where the first medical component and second, medical component are part of a single medical device.

13. The medical system of claim 6, where the first medical component comprises a first medical device and second medical component comprises a second medical device.

14. The medical system of claim 6, where the first medical component and second medical component are coupled via a joint member.

15. The medical system of claim 14, where the second medical component is moveable relative to the joint member.

16. The medical system of claim 15, where the second medical component is slidable over the joint member, such that the second medical component covers the flexible joint.

17. The medical system of claim 14, where the second medical component comprises a tether.

18. The medical system of claim 1, further comprising a first and second handles coupled the respective arm's proximal portion.

19. The medical system of claim 1, where the first medical component comprises a flexible member.

20. The medical system of claim 19, where the flexible member is hollow.

21. The medical system of claim 20, further comprising a medical component slidably located within the flexible member.

22. The medical system of claim 19, where the flexible member comprises a structure selected from the group consisting of a ribbon, a wire, and a coiled guide-wire.

23. The medical system of claim 19, further comprising a second medical component advanceable over the flexible member.

24. The medical system of claim 1, where the first medical component comprises at least one electrode.

25. The medical system of claim 1, where the first medical component comprises an energy delivery device.

26. The medical system of claim 25, where the energy delivery device comprises an energy modality selected from the group consisting of radiofrequency, DC, microwave, ultrasound, laser, cryogenic energy.

27. The medical system of claim 25, where the energy delivery device further comprises a tether extending from a distal portion.

28. The medical system of claim 1, further comprising at least a third arm, where the third arm comprises a grasping structure adapted to be releasbly coupled to a portion of the first medical component.

29. The medical system of claim 28, where the third arm comprises a maneuverable distal portion and a proximal portion, where manipulation of the third arm's proximal portion permits articulation of the third arm's maneuverable distal portion independently of the first and second arms.

30. The medical system of claim 28, where the third arm comprises at least one electrode on an outer surface.

31. The medical system of claim 28, where the grasping structure comprises a releasable hook, ring, or jaws.

32. The medical system of claim 1, where the first arm comprises a lumen extending therethrough, and where the medical component is advanceable from a proximal portion of the first arm coaxially through the first arm and out of the maneuverable distal portion of the first arm.

33. The medical system of claim 1, where the first medical component is coupled to a retaining section on the first arm's maneuverable distal portion, such that a proximal portion of the first medical component can move independently of the first arms' proximal portion.

34. The medical system of claim 1, where the first medical component is adapted to be temporarily affixed to the first, arm.

35. The medical system of claim 1, where the first medical component is adapted to be temporarily affixed to the second arm.

36. The medical system of claim 1, where at least the first arm is lockable such that the maneuverable distal portion is temporarily affixed into the profile.

37. The medical system of claim 1, where at least the first arm is comprises a plurality of steering members extending from the maneuverable distal portion to the proximal portion.

38. The medical component of claim 37, where the steering members are configured to move the first arm's maneuverable distal portion without rotating the first arm.

39. The medical system of claim 1, where at least the first arm comprises a varying degree of flexibility from the proximal portion to the maneuverable distal portion.

40. The medical system of claim 1, where the first arm comprises a first handle portion located at the first arm's proximal portion and having at least one steering mechanism to control articulation of the first arm's maneuverable distal portion.

41. The medical system of claim 1, where the second arm comprises a second handle portion located at the second arm's proximal portion and having at least one steering mechanism to control articulation of the second arm's maneuverable distal portion.

42. A medical system for performing a medical procedure on or in a patient, the system comprising:
a first medical component;
a first arm having a distal portion and a proximal portion, where manipulation of the first arm's proximal portion permits articulation of the first arm's distal portion and where the first medical component is coupled to and advanceable relative to the distal portion;
a second arm having a distal portion and a proximal portion, where manipulation of the second arm's proximal portion allows for articulation of the second arm's distal portion;
a flexible member coupled to the first medical component and extending through the second arm; and
where the first and second arm are configured to be manipulated independently, and where manipulation of the first or second arm alters a profile or position of the first medical component allowing for positioning of the first medical component.

43. The medical system of claim 42, where the flexible member is removable from the first or second arm to permit insertion of the arms into at least two separate openings in a tissue of the patient.

44. The medical system of claim 42, where the flexible member comprises a flexible tether and extends through to the second arm's proximal portion allowing for pulling of the first medical component from the second arm's proximal portion.

45. The medical system of claim 42, further comprising a second medical component adapted to engage and move along the flexible member and relative to the second arm's distal portion.

46. The medical system of claim 45, where the first medical component comprises a first coupling section and where the second medical component comprises a second coupling section, where the coupling sections are configured to nest, together such that the first and second medical components form a contiguous device.

47. The medical system of claim 42, further comprising a flexible joint between the flexible member and the first medical component, where a flexibility of the flexible joint is greater than that of the flexible member or first medical component.

48. The medical system of claim 47, where the flexible member is slidable relative to the flexible joint.

49. The medical system of claim 47, where the flexible member is slidable over the flexible joint, such that the tether member covers the flexible joint.

50. The medical system of claim 47, where the first arm comprises at least one electrode on an outer surface.

51. The medical system of claim 50, where the second arm comprises at least one electrode on an outer surface.

52. The medical system of claim 42, further comprising a second medical component extendable from the second arm's maneuverable distal portion.

53. The medical system of claim 52, where the second medical component comprises at least one electrode.

54. The medical system of claim 52, where the second medical component includes a second lumen extending therethrough, and where the second medical component is advanceable over a portion of the flexible member.

55. The medical system of claim 52, where the first medical component comprises a first mating portion and where the second medical component comprises a second mating portion, where the first and second mating portions releasably couple to each other.

56. The medical system of claim 42, further comprising a first and second handles coupled to the respective arm's proximal portion.

57. The medical system of claim 42, where the flexible member comprises a structure selected from the group consisting of a thread, a ribbon, a wire, a polymeric mono-filament, and a coiled guide-wire.

58. The medical system of claim 42, where the first medical component comprises at least one electrode.

59. The medical system of claim 42, where the first medical component comprises an energy delivery device.

60. The medical system, of claim 59, where the energy delivery device comprises an energy modality selected from the group consisting of radiofrequency, DC, microwave, ultrasound, laser, cryogenic, ultrasonic.

61. The medical system of claim 42, further comprising at least a third arm, where the third arm comprises a grasping structure adapted to be releasbly coupled to a portion of the first medical component, or the flexible member.

62. The medical system of claim 61, where the third arm comprises a maneuverable distal portion and a proximal portion, where manipulation of the third arm's proximal portion permits articulation of the third arm's maneuverable distal portion independently of the first and second arms.

63. The medical system of claim 61, where the third arm comprises at least one electrode on an outer surface.

64. The medical system of claim 61, where the grasping structure comprises a releasable hook, ring, or jaws.

65. The medical system of claim 42, where the first arm comprises a lumen extending therethrough, and where the first medical component is advanceable from the first arm's proximal coaxially through the first arm's maneuverable distal portion.

66. The medical system of claim 42, where the first medical component is coupled to a retaining section on the first arm's maneuverable distal portion, such that a proximal portion of the first medical component can move independently of the first arms' proximal portion.

67. The medical system of claim 42, where at least the first arm is lockable such that the maneuverable distal portion is temporarily affixed into the profile.

68. The medical system of claim 42, where at least the first arm is comprises a plurality of steering members extending from the maneuverable distal portion to the proximal portion.

69. The first medical component, of claim 68, where the steering members are configured to move the first arm's maneuverable distal portion without rotating the first arm.

70. The medical system of claim 68, where at least die first arm comprises a varying degree of flexibility from the proximal portion to the maneuverable distal portion.

71. A method for performing a procedure on tissue, the method comprising:
positioning at least a first and second arm adjacent to the tissue, where each arm includes a distal portion and a proximal portion, where manipulation of each arm's proximal portion causes movement of the respective arm's distal portion independently of the other arm;
advancing a medical component through the first arm to the tissue such that a portion of the medical component moves between the first and second arm;
manipulating either the first or second arm within a three dimensional area and independently of the other arm to alter a profile or location of the first, medical component; and
performing the procedure with the medical component.

72. The method of claim 71, where advancing a medical component through the first arm to the tissue couples the portion of medical component to the second arm.

73. The method of claim 71, where the first medical component is coupled to a flexible tether extending into the second arm's distal portion, and where manipulating either the first or second aim alters a shape of the flexible tether.

74. The method of claim 73, further comprising separating the flexible tether from the first medical component to expose a joint member, where the joint member couples the flexible tether to the first medical component.

75. The method of claim 71, further comprising advancing the first arm and medical component into the body and subsequently coupling the first medical component to the second arm.

76. The method of claim 71, further comprising advancing a second medical component through the second arm, and where performing the procedure further comprises using the second medical component to perform the procedure.

77. The method of claim 73, further comprising removably coupling the first and second medical components to alter the profile or location of the medical components.

78. The method of claim 71, where manipulating either the first or second arm comprises manipulating the respective arm in a three dimensional area without rotating the proximal portion of the arm.

79. The method of claim 71, where the first medical component comprises an energy delivery device, and where performing the medical procedure comprises delivering energy to tissue using the energy delivery device.

80. The method of claim 71, further comprising coupling a third, arm to the medical component where the third arm comprises a distal portion and a proximal portion, where manipulation of the third arm's proximal portion allows for positioning of the third arm's distal portion independent of the second arm.
